(19) United States
(12) Patent Application Publication
(10) Pub. No.: US 2009/0209829 A1
(43) Pub. Date: Aug. 20, 2009

(54) APPARATUS FOR DETECTING DRIVER'S MENTAL STATE AND METHOD FOR DETECTING MENTAL STATE

(75) Inventors: Masatoshi Yanagidaira, Saitama (JP); Mitsuo Yasushi, Saitama (JP)

Correspondence Address: DRINKER BIDDLE & REATH (DC), 1500 K STREET, N.W., SUITE 1100, WASHINGTON, DC, 20005-1209, US

(73) Assignee: PIONEER CORPORATION, TOKYO (JP)

(21) Appl. No.: 12/294,194

(22) PCT Filed: Mar. 22, 2007
(86) PCT No.: PCT/JP2007/055908

§ 371 Date: Mar. 30, 2009

(30) Foreign Application Priority Data
Mar. 24, 2006 (JP) 2006-083161

(51) Int. Cl. A61B 5/00; A61B 5/0402

(52) U.S. Cl. 600/301; 600/509

(57) ABSTRACT

The reliability of detection of a driver's mental state in a vehicle in a driving state is enhanced without impairing operation stability of driving action of the vehicle.

There are provided an imaging section 11 and a heart rate sensor 12 that serve as a plurality of detection means for detecting a driver's state; an appearance determination section 13 for determining a driver's appearance from detection results from the imaging section 11 and the heart rate sensor 12; and a mental state determination section 15 for determining a driver's mental state from the detection results from the imaging section 11 and the heart rate sensor 12. A switching section 14 switches the outputs from the imaging section 11 and the heart rate sensor 12 to an output from the mental state determination section 15 on the basis of a determination result from the appearance determination section 13.

### TECHNICAL FIELD

The present invention relates to a driver's mental state detection apparatus for detecting a mental state of a driver who operates a steering wheel of a vehicle, a ship, and the like, as well as to a mental state detection method.

### BACKGROUND ART

In order to prevent occurrence of an accident, which would otherwise be caused when a driver of a vehicle or a ship falls asleep while driving, it has hitherto been proposed to provide a steering-wheel with electrodes for measuring a driver's heart when hands of the driver remain in touch with the electrodes and to issue an alarm when an abnormal heart rate is measured (see; for instance, Patent Document 1).

According to the invention, only when both hands rather than a single hand touch the electrodes disposed on both sides of a steering wheel, heart rate data acquired from the electrodes of the steering wheel can be input to a CPU. When the heart rate is determined to be abnormal, an alarm system can be activated.

It has also been proposed to determine a driver's mental state from heart rate information about the driver during the travel of a vehicle and to issue a message for urging awakening by means of a video or an audio when the driver is in; for instance, a drowsy state (see; for instance, Patent Document 2).

According to the invention, a cardiogram is measured by means of a potential difference existing between both hands of the driver, and a heart rate is measured by means of a resultant waveform. The driver is determined to be either a drowsy state or a state of tension from measurement data, and a result of determination is messaged in the form of a video or an audio by means of an indicator or a speaker, thereby making it possible to prompt the driver to take a countermeasure.

In terms of use, above-mentioned detection of a heart rate is subjected to limitations; namely, a necessity to hold electrodes in both hands during measurement.

In the meantime, a driving state determination apparatus that is free from inconvenience in terms of handling and that uses a camera has hitherto been proposed (see; for instance, Patent Document 3).

The apparatus captures an image of a driver's face; detects driver's sight lines of the image by means of a sight line detector; computes a distribution of sight lines achieved within a predetermined period of time by sight line frequency distribution computing means; and determines a driver's distracted state (careless state) from the distribution of sight lines, and alarm means issues an alarm.

Patent Document 1: Japanese Patent No. 3727803

Patent Document 2: JP-A-2004-344269

Patent Document 3: Japanese Patent No. 3027786

### DISCLOSURE OF THE INVENTION

### Problem that the Invention is to solve

However, as described in Patent Document 1, it is difficult to consider a situation where a driver performs driving with one hand in a sleepy state or a state of tension from the viewpoint of a driver's ordinary driving action. On the contrary, such a drowsy state or a state of tension is induced by driving operation performed with two hands. Meanwhile, a state where one-handed driving is continually performed arises when the driver is relaxed or distracted.

Moreover, according to the descriptions of Patent Document 3, a distracted state can be detected on the basis of movements of driver's sight lines. A state where sight lines become stationary arises even when a vehicle is cruising down a freeway or a tunnel, and the technique cannot be said to be optimum as a method for detecting distraction.

As mentioned above, in the related art, it has been difficult to enhance the reliability of detection of a driver's mental state in a vehicle in a driving state, without impairing operation stability of driving action of the vehicle.

A problem to be solved by the present invention includes enhancement of reliability of detection of a driver's mental state in a vehicle in a driving state.

### Means for Solving the Problem

A mental state detection apparatus defined in claim 1 is characterized by comprising: a plurality of detection means for detecting a driver's state; appearance determination means for determining a driver's appearance from detection results from the detection means; mental state determination means for determining a driver's mental state from the detection results from the detection means; and switching means that switches the outputs from the detection means to an output from the mental state determination means on the basis of a determination result from the appearance determination means.

A mental state detection method defined in claim 15 is characterized by comprising: a detection step of detecting a driver's state by means of a plurality of detection means; an appearance determination step of determining a driver's appearance on the basis of detection results from the detection means; a mental state determination step of determining the driver's mental state on the basis of a detection result from the detection means by the mental state determination means; and a switching step of switching outputs from the detection means to an output from the mental state determination means on the basis of a result of determination made in the appearance determination step.

### BRIEF DESCRIPTION OF THE DRAWINGS

FIG. 1 A block diagram showing an apparatus for detecting a driver's mental state of an embodiment of the present invention.

FIG. 2 A flowchart showing a basic method for detecting a mental state of the embodiment of the present invention.

FIG. 3 A flowchart showing a specific method for detecting a mental state of the embodiment of the present invention.

FIG. 4 A flowchart showing another method for detecting a mental state of the embodiment of the present invention.

FIG. 5 A view for describing the configuration of a mental state detection apparatus for detecting a plurality of (N) mental states.

FIG. 6 A descriptive view showing a relationship between a driver's appearance, a driver's mental state and a sensor for detecting the appearance and the state.

FIG. 7 A flowchart showing procedures for estimating fatigue, a sense of a fret, tension, drowsiness, and a distracted state.

### DESCRIPTIONS OF THE REFERENCE NUMERALS

10 MENTAL STATE DETECTION APPARATUS
11 IMAGING SECTION (DETECTION MEANS)
12 HEART RATE SENSOR (DETECTION MEANS)
13 APPEARANCE DETERMINATION SECTION (APPEARANCE DETERMINATION MEANS)
14 SWITCHING SECTION (SWITCHING MEANS)
15 MENTAL STATE DETERMINATION SECTION (MENTAL STATE DETERMINATION MEANS)

### BEST MODE FOR IMPLEMENTING THE INVENTION

An embodiment of the present invention relates to a driver's mental state detection apparatus including a plurality of detection means for detecting a driver's state; appearance determination means for determining a driver's appearance from detection results from the detection means; mental state determination means for determining a driver's mental state from the detection results from the detection means; and switching means that switches the outputs from the detection means to an output from the mental state determination means on the basis of a determination result from the appearance determination means.

As a result, switching among detection results from the plurality of detection means is performed according to the driver's appearance. Compared with a case where a mental state is independently detected, the reliability of detection of a mental state can be enhanced further.

The driver's mental state include states, such as a sense of tension, drowsiness, a sense of a fret, a sense of fatigue, and carelessness (distraction), in which special attention is required to perform driving of a vehicle, or the like.

Electrodes for detecting a heart rate provided on a steering wheel handled by a driver are used as the mental state detection means. A driver's heart rate is measured by means of the driver touching the electrodes. When a normal heart rate is not acquired for a given period of time or more, driving can be determined to be performed with one hand.

When the steering wheel is held with both hands, tension or drowsiness can be determined to be an object of estimation from the viewpoint of driver's action. In the meantime, when the steering wheel is held with one hand, a relaxed state or a distracted state (a careless state) can be deemed to be an object of estimation.

The driver's appearance changes according to a driver's mental state and includes a “relaxed driving posture,” a “fatigued driving posture,” “another driving posture,” and the like. The “relaxed driving posture” is like driving a steering wheel with one hand while the driver puts his/her one hand on a window frame, and the “fatigued driving posture” is like a hunchback, a posture achieved when the driver throws back his/her head, and others.

When compared with a case where a mental state or a driver's appearance is independently detected as in the related art, the reliability of detection of a mental state can be enhanced by means of taking a mental state corresponding to the driver's appearance, such as that mentioned above, as an object of detection.

The mental state determination means performs mental state determination processing corresponding to an output from the detection means. As a result, the driver's mental state can be accurately ascertained in accordance with a predetermined mental state determination algorithm. According to the determination result, an alarm, for instance, is issued, to thus enable making of a contribution to safety driving.

In the embodiment, the switching means switches mental state determination processing to be performed by the mental state determination means, as well as switching the outputs from the detection means to an output from the mental state determination means. Therefore, processing for determining a driver's mental state can be practiced in a separated manner by means of switching of a mental state determination algorithm.

The mental state determination means includes first determination processing for determining a state of tension or drowsiness of the driver and second determination processing for determining a distracted state of the driver. As a result, by means of the first determination processing and the second determination processing switched by the switching means, determination of a state of tension or drowsiness and determination of a careless state can be separately practiced.

The detection means has heart rate measurement means for measuring a heart rate of the driver by means of electrodes provided on the steering wheel of a vehicle and imaging means for imaging the driver. By means of the configuration, a driver's mental state conforming to the current heart rate and dynamics of the driver can be determined.

In the present embodiment, the first determination processing is configured such that the state of tension or drowsiness of the driver is determined from heart rate information obtained from the heart rate measurement means, and the second determination processing is configured such that a distribution of directions of sight lines of the driver is examined on the basis of imaging information about the driver obtained from the imaging means provided in a compartment, to thus determine a driver's distracted state from information about the distribution. Thereby, the state of tension or drowsiness of the driver can be determined from heart rate information, and the distracted state of the driver can be accurately determined from imaging information.

In the embodiment, the appearance determination means is configured so as to determine, when normal heart rate information is input from the heart rate measurement means longer than a predetermined period of time, that the driver holds the steering wheel with both hands and to determine, when the normal heart rate information is not input longer than the predetermined period of time, that the driver holds the steering wheel with one hand. As a result, driver's tension or drowsiness can be estimated as an object while the driver holds the steering wheel with both hands, and a driver's distracted state can be estimated as an object while the driver holds the steering wheel with one hand.

The switching means is configured so as to perform switching, when the appearance determination means determines that the steering wheel is held with both hands, such that the heart rate information acquired from the heart rate measurement means is output to the mental state determination means and to perform switching, when the appearance determination means determines that the steering wheel is held with one hand, such that the imaging information acquired from the imaging means is output to the mental state determination means.

Thereby, the mental state determination means can determine the driver's mental state achieved when the driver holds a steering wheel with both hands while taking a driver's state of tension or distraction as an object. Further, the driver's mental state achieved when the driver holds a steering wheel with one hand can be determined while a distracted mental state of the driver is taken as an object.

In the present embodiment, the mental state determination means includes third determination processing for determining whether the driver assumes a hunchback posture or a posture achieved when the driver throws back his/her head. By means of the configuration, a driver's mental state, such as fatigue or a fret, can be analyzed on the basis of a distribution of pressure over a back or hip area of a driver seat through the third determination processing.

In the present embodiment, the third determination processing is configured so as to examine a distribution of driver's body pressure by means of an output from a pressure sensor disposed in the driver seat and determine driver's fatigue or a fret from information about the distribution. As a result, the driver's fatigue or a fret can be simply, inexpensively determined from the output from the pressure sensor.

The mental state determination means includes fourth determination processing for determining the frequency of deep breathing performed by the driver. Thereby, the driver's mental state, such as a fret, fatigue, or tension, can be determined by means of the fourth determination processing.

In the fourth determination processing, the frequency of occurrence of a deep breath is examined on the basis of the imaging information about the driver acquired from the imaging means provided in the compartment, and the driver's state of fatigue, a fret, or tension is determined from the frequency of occurrence. Thereby, the driver's state of fatigue, a fret, or tension can be accurately determined by means of processing the imaging information.

The mental state determination means includes fifth determination processing for determining vibrations in the driver's head. Therefore, the drowsy and distracted states of the driver can be estimated from the degree of awareness acquired through the fifth determination processing.

In the fifth determination processing, the state of vibrations in the driver's head is examined on the basis of the imaging information about the driver acquired from the imaging means provided in the compartment, thereby determining the degree of driver's awareness from the number of vibrations or a vibration level. Thereby, the drowsy and distracted states of the driver can be estimated from the determined degree of awareness.

The present embodiment includes a detection step of detecting a driver's state by means of a plurality of detection means; an appearance determination step of determining a driver's appearance on the basis of detection results from the detection means; a mental state determination step of determining the driver's mental state on the basis of the detection results from the detection means by the mental state determination means; and a switching step of switching outputs from the detection means to an output from the mental state determination means on the basis of a determination result acquired in the appearance determination step.

Thereby, the detection results from the plurality of detection means can be output in a switched manner in accordance with the driver's appearance. When compared with a case where a mental state is independently detected, the reliability of detection of a mental state can be enhanced, and action responsive to the driver's mental state; for instance, issuance of an alarm, can be performed, to thus enable making of a contribution to safety driving.

### EXAMPLES

Specific examples of an apparatus for detecting a driver's mental state of the embodiment of the present invention will now be described in detail by reference to the drawings.

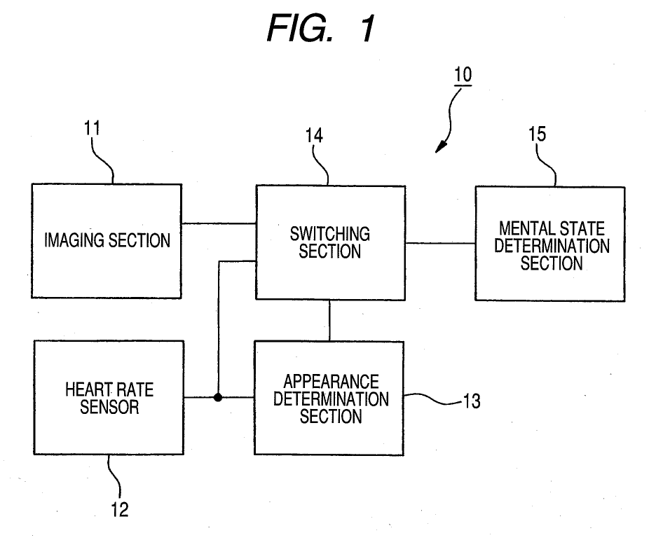

FIG. 1 is a block diagram showing an apparatus for detecting a driver's mental state of the embodiment. A mental state detector 10 has an imaging section 11; a heart rate sensor 12; a driver's appearance determination section 13; a switching section 14; and a driver's mental state determination section 15.

The imaging section 11 captures an image of a face and sight lines of a driver by means of a camera disposed in a compartment. The heart rate sensor 12 is provided on a steering wheel held and operated by the driver. When the driver touches the steering wheel with both hands, a driver's heart rate is measured.

When falling into a state of tension or a drowsy state, the driver feels a danger. Therefore, there are many cases where even a driver who usually has a habit of operating a steering wheel with one hand unconsciously holds the steering wheel with both hands. When performing driving operation in a relaxed driving posture, the driver can enter a distracted state. Moreover, when performing driving operation in a fatigued driving posture, the driver often feels a sense of a fret or fatigue.

The appearance determination section (appearance determination means) 13 determines the driver's appearance according to heart rate information from the heart rate sensor 12.

In accordance with the driver's appearance determined by the appearance determination section 13, the switching section (switching means) 14 acts so as to switch an output from the imaging section 11 or the heart rate sensor 12 to an output from the mental state determination section 15 and switch mental state determination processing performed by the mental state determination section 15. The mental state determination section (mental state determination means) 15 acts so as to perform mental state determination processing according to the information switched by the switching section 14.

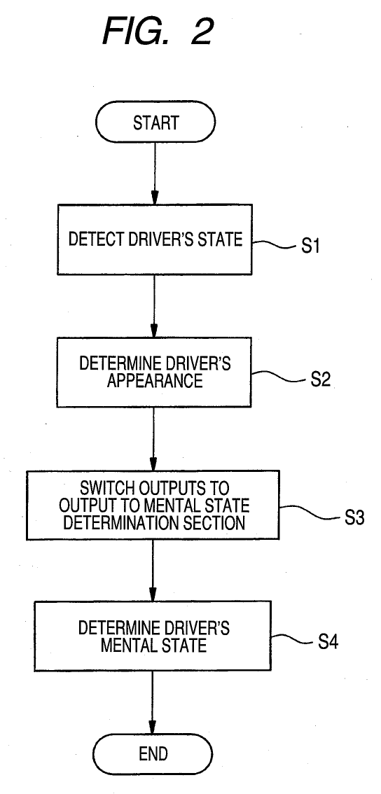

A method for detecting a driver's mental state will now be described by reference to a flowchart shown in FIG. 2.

First, a plurality of detection means (the imaging section 11 and the heart rate sensor 12 in this case) detect driver's conditions (step S1), whereas a determination is made as a driver's appearance, on the basis of a detection result from the detection means (the heart rate sensor 12), as to whether or not the driver holds the steering wheel with both hands (step S2). In accordance with a result of determination of the appearance, outputs from the detection means (the imaging section 11 and the heart rate sensor 12) are switched to an output from the mental state determination section 15 (step S3), and the mental state determination section 15 determines, on the basis of detection results from the detection means (the imaging section 11 and the heart rate sensor 12), the driver's mental state (step S4).

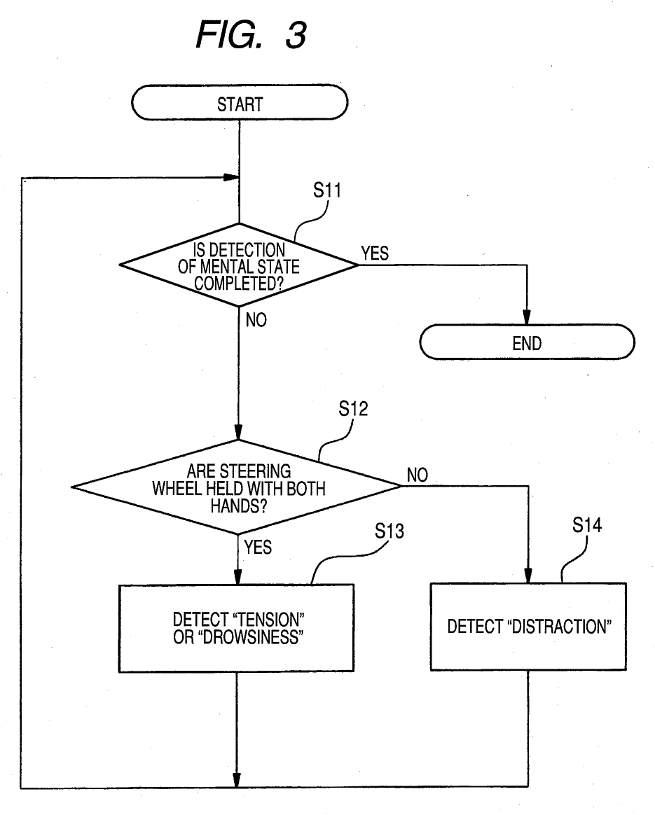

Next, a specific example of the mental state detection method will be described by reference to FIG. 3. First, detection of a driver's mental state is commenced. It is determined, as a driver's appearance, whether or not the driver holds the steering wheel with both hands (step S12) until detection of a mental state is completed (step S11). The appearance determination section 13 performs a determination on the basis of the heart rate data from the heart rate sensor 12.

When determined that a driver's heart rate cannot be normally acquired from electrodes disposed on the steering wheel for a given period of time or longer, the appearance determination section 13 determines that the driver holds the steering wheel with one hand and performs switching to distraction information acquired from the imaging section 11, whereupon the mental state determination section 15 determines the driver's mental state as a state of distraction (step S14).

When the driver is determined to hold the steering wheel with both hands, the mental state is switched in accordance with an output from the appearance determination section 13; namely, switching is made to information about a change in heart rate, whereby the driver's mental state is determined (presumed) to be tension or drowsiness (step S13).

Thus, the switching section 14 performs switching, to thus detect a mental state conforming to the driver's appearance, thereby yielding an advantage of enhancement of the reliability of detection of a mental state compared with the case where a mental state is solely detected.

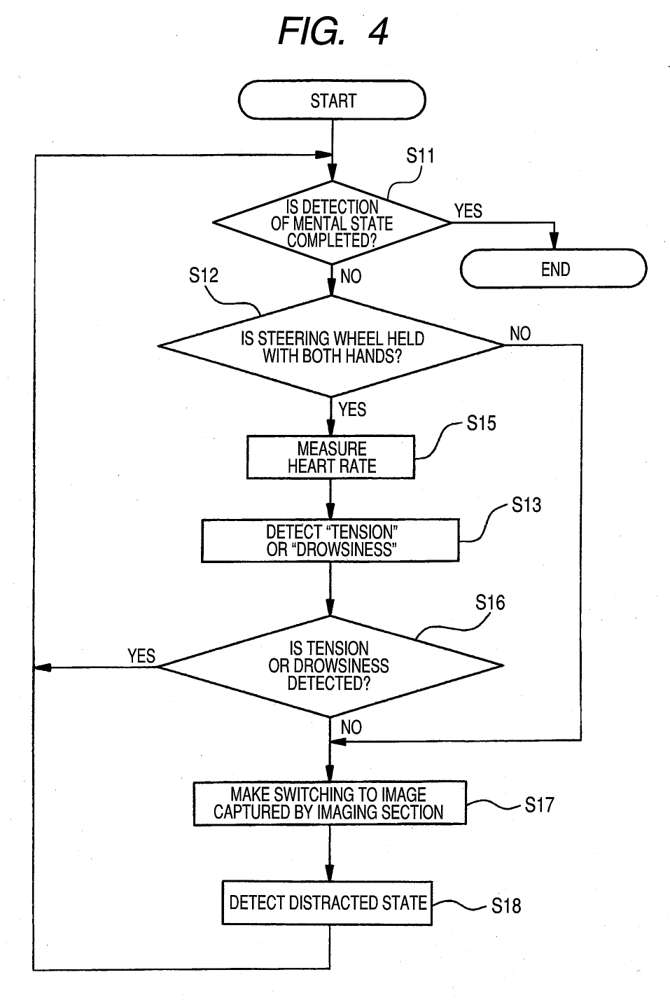

A state of holding of a steering wheel achieved when the driver is distracted is learned in consideration of a personal characteristic, whereby conditions for holding a steering wheel employed at the time of detection of a distracted state can be individually changed. As shown in FIG. 4, in this case, there is also a case where a distracted state is detected during the course of the steering wheel being held with both hands. Detection may also be performed by imposing limitations on a time except a time during which drowsiness or tension is detected.

In this case, when the driver is determined to hold the steering wheel with both hands according to the information output from the heart rate sensor 12 (step S12); the heart rate sensor 12 measures a heart rate (step S15); and tension or drowsiness, such as that mentioned above, is detected from a heart rate (step S13). Accordingly, it is examined whether or not tension or drowsiness is detected (step S16). When tension or drowsiness still remains undetected, switching is made to the image captured by the imaging section 11 (step S17), thereby detecting a distracted state (step S18).

When the handle is determined to be held with one hand in step S12, switching is made to the image captured by the imaging section 11, thereby detecting a driver's distracted state (step S17). A determination is made, on the basis of the captured image, as to whether or not the driver is in a distracted state (step S18). Thereby, the accuracy of detection of a distracted state can be enhanced without regard to an individual difference.

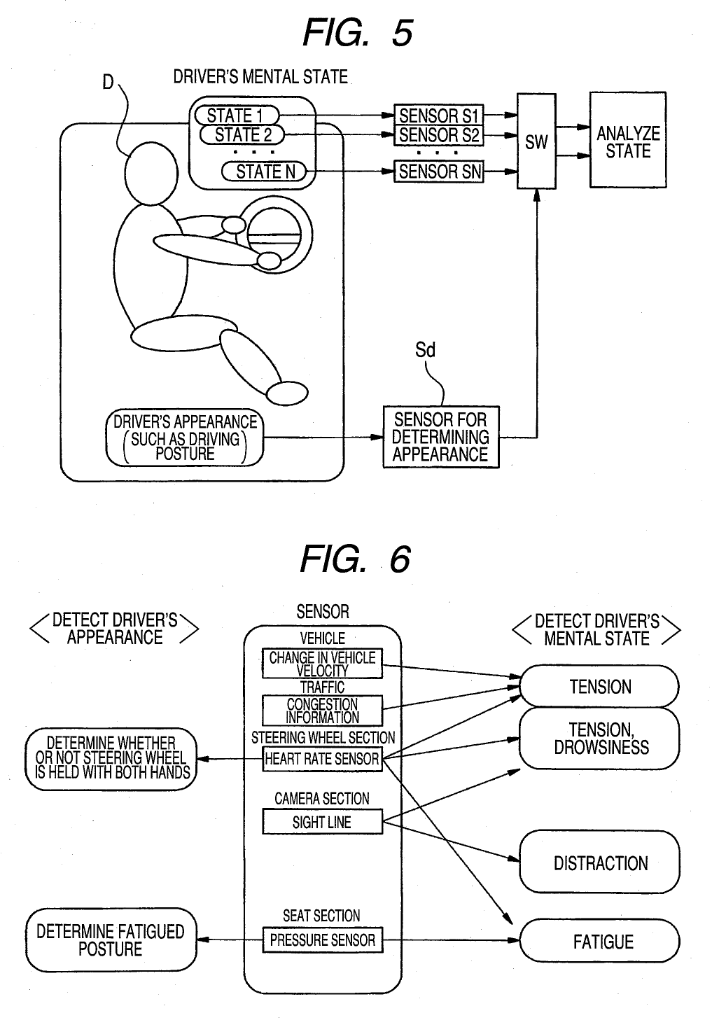

FIG. 5 is a view for describing the configuration of the mental state detection apparatus for detecting a plurality of (N) mental states. In the drawings, respective sensors S1 through SN detect respective mental states (State 1 through State N) of a driver D; namely, respective mental states, such as drowsiness, tension, distraction, a fret, and fatigue, as one sensor detects one mental state. In the meantime, the appearance determination sensor Sd determines the appearance (a driving posture, or the like) of the driver D, and a mental state acquired from any one of the sensors S1 to SN switched by the switching means SW is analyzed in accordance with the determination result.

FIG. 6 is a descriptive view showing a relationship between a driver's appearance, a driver's mental state and the sensors for detecting the appearance and the state. As shown in FIG. 6, a determination as to whether or not the steering wheel is held with both hands, among determinations as to the driver's appearance, is detected by the heart rate sensor 12 disposed on the steering wheel, and determination of the fatigued driving posture is detected by a pressure sensor disposed in a seat section.

Moreover, in relation to determination of a driver's mental state, a fret can also be presumed from vehicle velocity, a change in vehicle velocity, information about traffic congestion, and the like, as well as from heart rate information from the heart rate sensor 12. Tension or drowsiness can be assumed from sight line distribution information acquired from the heart rate sensor 12 and the imaging section 11, and a distracted state can also be presumed from the sight line distribution information acquired from the imaging section 11. Fatigue can be presumed from pressure information acquired from the heart rate sensor 12 and the pressure sensor of the seat section.

The mental state detection means provided in the seat section uses a thin-seat-like pressure sensor (a body pressure distribution sensor) placed in the sheet section. A fatigued posture is determined by examining the distribution of pressure on the back or hip of the driver by means of the pressure sensor or determining whether the driver assumes a hunchback posture or throws back his/her head. When the fatigued driving posture is assumed, an analysis is performed while the mental state, such as fatigue or a fret, is taken as an object. In relation to estimation of fatigue attributable to long-hour driving, a method for observing a chronological change in the center of a load stemming from a change in posture, or a like method, is adopted.

In addition to including those mentioned above, the driver's appearance also includes body movements. The body movements entail deep breathing and head movements performed during driving operation. When the frequency of body movements is at a given level or more, occurrence of a fret, fatigue, or tension can be presumed. The reason for this is that body movements induced by deep breathing or breathing having a large amplitude, such as a sigh, are considered to be induced by a mental state, such as a fret, fatigue, and tension.

Vehicle velocity or a change in vehicle velocity is presumed in association with a change in heart rate as a method for estimating a state of a fret, as mentioned above. Specifically, a condition in which driving is intermittently performed at low speed is deemed to be a state of congestion. When an increase exists in heart rate at this time, the driver is presumed to feel frustration with congestion. In addition, traffic information acquired from a car navigation system may also be utilized for determining congestion.

Vibrations in the head synchronism with vibrations in vehicle may also be checked as a method for determining body movements. The reason for this is that, when the degree of awakening is higher than an ordinary level, the entire body of the driver is stiffened by means of putting power into muscles of individual parts of the body, whereby vibrations in head synchronized with vibrations in vehicle are reduced. In particular, the head has a high degree of freedom of movement, and hence such a difference is likely to appear. Therefore, when the vibrations are detected for a given period of time or longer, the degree of awakening is determined to have reduced, so that a drowsy state or a distracted state can be presumed.

Procedures for presuming a state of fatigue, a fret, tension, drowsiness, and distraction will now be described as another embodiment.

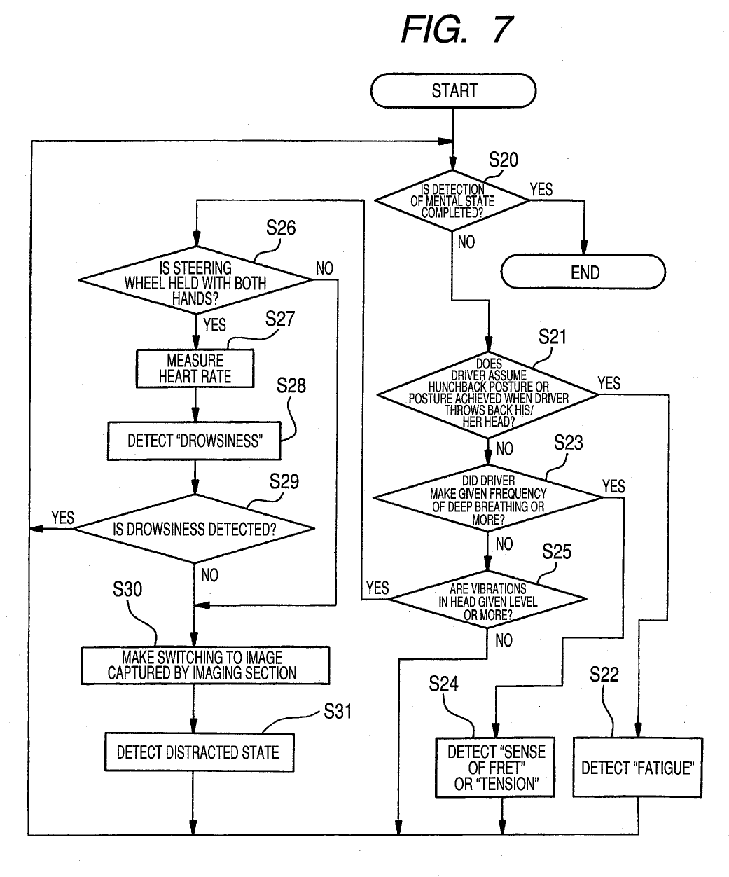

FIG. 7 is a view showing a flow along which a state of fatigue, a fret, tension, drowsiness, or distraction is presumed. As shown in FIG. 7, it is examined, on the basis of a pressure data distribution acquired from the pressure sensor disposed in the seat (the driver seat), whether or not the driver's posture is hunchback or a posture achieved when the driver throws back his/her head; namely, whether or not the driver assumes a fatigued posture (step S21), until detection of a mental state is completed (step S20). When the driving posture of the driver is hunchback or a posture achieved when the driver throws back his/her head (YES), a fatigued state of the driver is detected (step S22).

When the driving posture of the driver is determined not to be hunchback or the posture achieved when the driver throws back his/her head (NO), it is examined whether or not the frequency of deep breathing captured by the imaging section 11 is a given number of times or more (step S23). When the frequency of deep breathing is a given number of times or more (YES), occurrence of deep breathing is detected as a state of a fret or tension (step S24).

When the frequency of deep breathing is determined not to be a given number of times or more (NO: less than a given number of times), another examination is continually made as to whether or not vibrations in the driver's head are equal to a given amount or more (step S25).

When a result achieved in step S25 shows that the head vibrations are given amounts or more (YES), processing pertaining to procedures analogous to the flow (steps S12 to S18) shown in FIG. 4 is performed.

Specifically, when the steering wheel is determined to be held with both hands in accordance with the output information from the heart rate sensor 12 (step S26), the heart rate sensor 12 measures a heart rate (step S27), thereby detecting drowsiness, such as that mentioned above, from the heart rate (step S28). Accordingly, it is examined whether or not drowsiness is detected (step S29). When drowsiness still remains undetected, switching is made to the image captured by the imaging section 11 (step S30), and a state of distraction is detected (step S31).

When the head vibrations are determined to be less than given amounts (NO) in step S25, an ordinary mental state is determined. Thus, an object of detection of a driver's mental state is changed according to body movements that correspond to the driver's appearance.

In response to the thus-detected respective mental states, stability of operation for driving a vehicle can be ensured by means of generating; for instance, an alarm sound to prompt awaking, comfortable sound, or changing vehicle velocity, and the like.

As mentioned in detail, the driver's mental state detection apparatus 10 of the embodiment has the imaging section 11 and the heart rate sensor 12 that are a plurality of detection means for detecting a state of a driver; the appearance determination section 13 for determining a driver's appearance on the basis of a detection result from the heart rate sensor 12; and the mental state determination section 15 for determining a driver's mental state from detection results from the detection means. The switching section 14 switches outputs from the imaging section 11 and the heart rate sensor 12 to an output from the mental state determination section 15 on the basis of a determination result of the appearance determination section 13.

As a result, detection results from the plurality of detection means (the imaging section 11 and the heart rate sensor 12) can be output in a switched manner in accordance with the driver's appearance. Hence, when compared with the case where a mental state and driver's appearance are individually detected, the reliability of detection of a mental state can be enhanced. Therefore, the reliability of detection of a driver's mental state achieved in a vehicle in a traveling state can be enhanced without impairing stability of operation for driving a vehicle.

The method for detecting a driver's mental state of the present embodiment includes a detection step of detecting a driver's state by means of a plurality of detection means (the imaging section 11 and the heart rate sensor 12); an appearance determination step of determining a driver's appearance on the basis of detection results from the detection means (the imaging section 11 and the heart rate sensor 12) by means of the appearance determination section 13; a mental state determination step of determining the driver's mental state on the basis of the detection result from the detection means (the imaging section 11 and the heart rate sensor 12) by the mental state determination section 15; and a switching step of switching, on the basis of a result of determination made in the appearance determination step, outputs from the detection means (the imaging section 11 and the heart rate sensor 12) to an the mental state determination section 15.

Thereby, the detection results from the plurality of detection means (the imaging section 11 and the heart rate sensor 12) can be output in a switched manner in accordance with the driver's appearance. When compared with a case where a mental state is independently detected, the reliability of detection of a mental state can be enhanced, and action responsive to the driver's mental state; for instance, issuance of an alarm, can be performed, to thus enable making of a contribution to safety driving.

1. A mental state detection apparatus comprising:
a plurality of detection units configured to detect a driver's state;
an appearance determination unit configured to determine a driver's appearance from the detection results of the detection units;
a mental state determination unit configured to determine a driver's mental state from the detection results of the detection units; and
switching unit configured to switch the outputs from the detection units to the mental state determination unit on the basis of a determination result of the appearance determination unit.

2. The mental state detection apparatus according to claim 1, wherein the mental state determination unit performs mental state determination processing based on outputs from the detection units.

3. The mental state detection apparatus according to claim 1, wherein the switching unit switches the outputs from the detection units to the mental state determination unit and switches mental state determination processing performed by the mental state determination unit.

4. The mental state detection apparatus according to claim 1, wherein the mental state determination unit performs first determination processing for determining a state of tension or drowsiness of the driver and second determination processing for determining a distracted state of the driver.

5. The mental state detection apparatus according to claim 1, wherein the detection units include heart rate measurement unit that is configured to measure a heart rate of the driver by means of electrodes disposed in a steering wheel section of a vehicle and imaging unit that is configured to capture an image of the driver.

6. The mental state detection apparatus according to claim 5, wherein the first determination processing is for determining a state of tension or drowsiness of the driver in accordance with heart rate information from the heart rate measurement unit, and the second determination processing is for examining a distribution of directions of sight lines of the driver on the basis of imaging information about the driver from the imaging unit disposed in a compartment, to thus determine a distracted state of the driver on the basis of information about the distribution.

7. The mental state detection apparatus according to claim 5, wherein the appearance determination unit is configured so as to determine, when normal heart rate information is input from the heart rate measurement units longer than a predetermined period of time, that the driver holds the steering wheel with both hands and to determine, when the normal heart rate information is not input longer than the predetermined period of time, that the driver holds the steering wheel with one hand.

8. The mental state detection apparatus according to claim 7, wherein the switching unit is configured so as to perform switching, when the appearance determination unit determines that the steering wheel is held with both hands, such that the heart rate information acquired from the heart rate measurement units is output to the mental state determination unit and to perform switching, when the appearance determination unit determines that the steering wheel is held with one hand, such that the imaging information acquired from the imaging unit is output to the mental state determination unit.

9. The mental state detection apparatus according to claim 1, wherein the mental state determination unit performs third determination processing for determining whether the driver assumes a hunchback posture or a posture achieved when the driver throws back his/her head.

10. The mental state detection apparatus according to claim 9, wherein the third determination processing includes examining a distribution of driver's body pressure by of an output from a pressure sensor disposed in the driver seat and determining driver's fatigue or a fret from information about the distribution.

11. The mental state detection apparatus according to claim 1, wherein the mental state determination unit includes fourth determination processing for determining the frequency of deep breathing performed by the driver.

12. The mental state detection apparatus according to claim 11, wherein the fourth determination processing includes examining the frequency of occurrence of a deep breath on the basis of the imaging information about the driver acquired from the imaging unit provided in the compartment and determining the driver's state of fatigue, a fret, or tension from the frequency of occurrence.

13. The mental state detection apparatus according to claim 1, wherein the mental state determination unit performs fifth determination processing for determining vibrations in the driver's head.

14. The mental state detection apparatus according to claim 13, wherein the fifth determination processing includes examining the state of vibrations in the driver's head on the basis of the imaging information about the driver acquired from the imaging unit provided in the compartment, thereby determining the degree of driver's awareness from the number of vibrations or a vibration level.

15. A mental state detection method comprising:
detecting a driver's state by a plurality of detection units;
determining a driver's appearance on the basis of detection result of the detecting step;
determining a driver's mental state on the basis of the detection result of the detecting step by a mental state determination unit; and
of switching outputs from the detection units to the mental state determination unit on the basis of the determining result of the driver's mental state determining step.
